(19) United States
(12) Patent Application Publication
(10) Pub. No.: US 2020/0268303 A1
(43) Pub. Date: Aug. 27, 2020

(54) UROFLOWMETRY AND FECAL FLOWMETRY SYSTEM

(71) Applicant: Consortia Medical, LLC, Austin, TX (US)

(72) Inventor: Jeffrey A. Oliva, Driftwood, TX (US)

(21) Appl. No.: 16/618,351

(22) PCT Filed: May 31, 2018
(86) PCT No.: PCT/US18/35497

§ 371 Date: Nov. 30, 2019

Related U.S. Application Data
(60) Provisional application No. 62/512,789, filed on May 31, 2017.

(51) Int. Cl. A61B 5/20; A61B 10/00; A61B 5/00; G01F 1/66

(52) U.S. Cl. CPC A61B 5/208; A61B 10/0038; E03D 11/02; A61B 5/6891; G01F 1/661; A61B 5/0022

(57) ABSTRACT

A system comprising: a) a toilet comprising a toilet bowl for receiving excreta; b) a detection unit mounted to the toilet bowl and comprising: (i) one or more detectors selected from a group consisting of one or more laser triangulation sensors, an infrared sensing device and a Doppler velocimeter), (ii) a data processor, (iii) a data storage device, (iv) a wireless transmitter to transmit data from the detectors to a communications network and (v) a power source, wherein the one or more sensors are positioned measure characteristics of excreta deposited in the toilet bowl; and c) a computer configured to receive the data over the communications network and comprising memory including computer code to analyze the data and determine an amount of urine and/or feces deposited into the toilet bowl, and a processor to execute the code.

### REFERENCE TO RELATED APPLICATIONS

This application claims the benefit of the priority date of U.S. Provisional application 62/512,789, filed May 31, 2017, the contents of which are incorporated herein in their entirety.

### STATEMENT AS TO FEDERALLY SPONSORED RESEARCH

None.

### BACKGROUND

Uroflowmetry is the measurement of the rate of urine flow over time. It is a basic, initial step in the differential diagnosis of voiding dysfunctions. Certain conditions can affect a person's normal urine flow. These conditions include: benign prostatic hypertrophy, or enlargement of the prostate gland, which can block the urethra completely; bladder cancer; prostate cancer; a urinary blockage; neurogenic bladder dysfunction, or trouble with the bladder due to a nervous system problem such as spinal cord tumor or injury; and frequent urinary tract infections.

Doctors may recommend a uroflow test if the patient has slow urination or difficulty urinating. Doctors may also use the test to determine how well the urinary tract and sphincter muscle are functioning. The sphincter muscle is a circular muscle that closes tightly around the bladder opening. It helps to prevent urine leakage.

Conventional uroflowmetry systems or uroflowmeters typically include a toilet chair, a funnel, and a specially programmed scale that measures volume and flow rate. During the test, the patient can urinate into a funnel-shaped device or a special toilet for the uroflow test. The patient should urinate as he or she normally would, without attempting to manipulate the speed or flow in any way. Conventional methodologies are messy, may require frequent specimen handling, frequent cleaning, and cannot be easily performed outside of a clinic.

Similarly, detection of quantity and type of feces voided can be useful in the diagnosis of digestive and bowel function. This has been done by collecting feces in a container and examining the contents of the container.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an exemplary toilet based diagnostic system comprising a toilet and a detection unit.

FIGS. 2A and 2B show elements of an exemplary detection unit of this disclosure.

FIG. 3 shows exemplary path of dataflow.

FIG. 4 shows an exemplary method for processing infrared data captured by the uroflowmeter system of FIG. 1.

FIG. 5 shows an exemplary computer system.

FIG. 6 depicts an exemplary process involving making measurements on excreta by both infrared video and LDV, and transmission of the data generated to a server.

FIG. 7 depicts an exemplary process of analyzing data provided to a server and generating reports including, e.g., a uroflow graph and a Bristol stool report.

FIG. 8 shows a flow chart of steps in an algorithm to analyze excreta data produced by the systems of this disclosure.

FIG. 9 depicts an exemplary algorithm for analyzing excreta data.

FIG. 10 depicts an exemplary system using laser triangulation to determine shape and volume of excreta.

### SUMMARY

In one aspect, provided herein is a system comprising: a) a toilet comprising a toilet bowl for receiving excreta; b) a detection unit mounted to the toilet bowl and comprising: (i) one or more detectors selected from a group consisting of one or more laser triangulation sensors, an infrared sensing device and a Doppler velocimeter, (ii) a data processor, (iii) a data storage device, (iv) a wireless transmitter to transmit data from the infrared sensing device to a receiver that is connected to a communications network, and (v) a power source, wherein the one or more detectors are positioned measure characteristics of excreta deposited in the toilet bowl; and c) a computer configured to receive the data over the communications network and comprising memory including computer code to analyze the data and determine an amount of urine and/or feces deposited into the toilet bowl, and a processor to execute the code. In one embodiment the toilet is a sitting toilet or a squat toilet. In another embodiment the excreta comprises urine and/or feces. In another embodiment the detection unit is mounted to the bowl by means of a hook, a clamp, a hanger, a suction cup, a magnet, a bolt, a screw or a bracket. In another embodiment the detector comprises one or more laser triangulation sensors. In one embodiment of the one or more laser triangulation sensors comprise one or more 2D or 3D laser triangulation scanners. In another embodiment the detector comprises an infrared sensing device. In another embodiment the detector comprises a laser Doppler velocimeter. In another embodiment the detector comprises an infrared sensing device and a laser Doppler velocimeter. In another embodiment the detector comprises one or more laser triangulation sensors and a laser Doppler velocitometer. In another embodiment the data storage device comprises instructions for converting a signal from the infrared/laser sensing device for transmission by the wireless transmitter. In another embodiment the data processor executes instructions from the data storage device for converting a signal from the infrared/laser sensing device for transmission by the wireless transmitter. In another embodiment the wireless is configured for short link radio communication (e.g., Bluetooth); communication over a wireless local area network (e.g., Wi-Fi), communication over a cell phone or cellular network). In another embodiment the power source comprises a battery. In another embodiment the amount of urine and/or feces is determined as a function of heat measurements included in the data. In another embodiment the amount of urine and/or feces is determined as a function of 2-D or 3-D shape of excreta. In another embodiment the computer code analyzing the data analyzes a video thermal image in which urine is detected at a first temperature in feces is determined at the second temperature. In another embodiment the computer code analyzes the data detects urine and/or feces as a function of temperature above ambient temperature. In another embodiment, the computer code analyzing the data detects urine and/or feces as a function of velocity and/or displacement. In another embodiment the computer code analyzing the data detects urine and/or feces as a function of shape and/or density of excreta. In another embodiment the computer code analyzing the data analyzes data collected over a predetermined time, e.g., about one hour to about one day, about one day to two weeks, about one week to about one month, about one month to about one year.

In another aspect provided herein is an excreta flow detection and reporting unit comprising: (i) one or more detectors selected from a group consisting of one or more laser triangulation sensors, an infrared sensing device and/or a laser Doppler velocimeter; (ii) a data processor; (iii) a data storage device; (iv) a wireless transmitter to transmit data from the infrared sensing device to a receiver that is connected to a communications network; (v) a power source, and, optionally, (vi) a support for mounting the unit in a toilet bowl. In one embodiment the support comprises a clamp, a hanger, a hook, a suction cup, a magnet, a bolt, a screw, or a bracket.

In another aspect provided herein is a method comprising: a) detecting, with one or more detectors, excreta data from a toilet bowl by laser triangulation sensing, infrared sensing, Doppler velocity sensing, or a combination thereof; b) wirelessly transmitting the data to a communications network; c) receiving the data transmitted to the communications network into a server; and d) analyzing the data to determine the amount of excreta deposited in the toilet bowl. In one embodiment the detecting further comprises activating the one or more detectors to collect data upon detection of motion with a motion sensor and deactivating the one or more detectors upon detection of cessation of motion with the motion sensor, wherein the motion is associated with excreta is being deposited in the toilet bowl. In another embodiment the detectors comprise an infrared video camera and a laser Doppler velocimeter, and analyzing the data comprises one or more of (i) duration of excretion (e.g., based at least in part on the first time in last time of detection of excreta through a data capture field), (ii) flow rate (e.g., based on volume per unit of time); (iii) flow volume (e.g., as a function of duration and flow rate); and (iv) class of excreta (e.g., urine or feces, e.g., based on density and/or shape of excreta). In another embodiment the detectors comprise one or more laser triangulation sensors, and a Doppler velocitometer, analyzing the data comprises: determining three-dimensional shape and volume of excreta using data from the laser triangulation sensors; determining density of the excreta using Doppler velocitometer data; categorizing excreta as urine or feces based on density and/or shape data; and determining volume and/or type of excreta based on shape and/or volume data and density data.

In another embodiment the method further comprises: e) generating a report indicating an amount and pattern of urine and/or feces deposited into the toilet bowl over a specified period of time. In another embodiment the report comprises a uroflow graph and/or a Bristol stool report.

In another aspect provided herein is a system comprising: a) a communication interface (e.g., a network interface card, network adapter, LAN adapter or physical network interface) that receives, over a communications network, laser triangulation sensing data, infrared sensing data, Doppler velocity sensing data, or a combination thereof; and b) a computer in communication with a communications interface, wherein the computer comprises one or more processors and a computer readable medium comprising machine executable code in tangible form that, upon execution by the one or more computer processors, implements a method comprising: i) receiving the data, over the communications network; ii) executing the machine executable code to determine volume and type of excreta, optionally including timing of excretion; and, optionally, iii) transmitting the determinations over the communications network, e.g., to a remote computer.

Advantages of the system may include one or more of the following: The system provides uroflowmetry with improved accuracy, cleanliness, and ease of conducting the test within and outside of a clinic setting. In addition, the system allows for fecal flowmetry measurement all within any normal toilet. This system will also enable cost effective, remote measuring and monitoring of toileting patterns in the elderly population, allowing for clinical intervention as appropriate. This includes early identification of dehydration risk for intervention.

### DETAILED DESCRIPTION

### I. Introduction

Disclosed herein are systems and methods for measuring excreta, such as urine and feces. An exemplary system includes a toilet having a detection unit attached. The detection unit captures data from the toilet relating to material voided by a subject (“voided material” or “excreta”) and deposited into the toilet. The data can comprise, for example, laser triangulation images, infrared images and/or laser Doppler data. The detection unit wirelessly transmits the captured data to a receiver. The receiver, in turn, transmits the data over a communications network to a central computer. A software application accesses the data in the central computer and executes an algorithm that analyzes the data to generate information. The information can include, for example, total volume or mass of excreta, timing of voiding functions or quality features of excreta. A medical professional, such as a doctor or nurse, can review the information and advise the subject or a medical professional treating the subject about the meaning and/or implications of the information. This can include, for example, whether the subject is dehydrated, constipated or suffering from chronic diarrhea or overactive bladder. Therapeutic interventions can be taken to address issues found, such as changing eating or drinking habits, wearing incontinence products such as pads or undergarments, or surgical intervention.

### II. Toilet System

A. Toilet

Toilet systems of this disclosure comprise a toilet and a detection unit attached to the toilet and positioned to detect excreta deposited by a user into the toilet bowl.

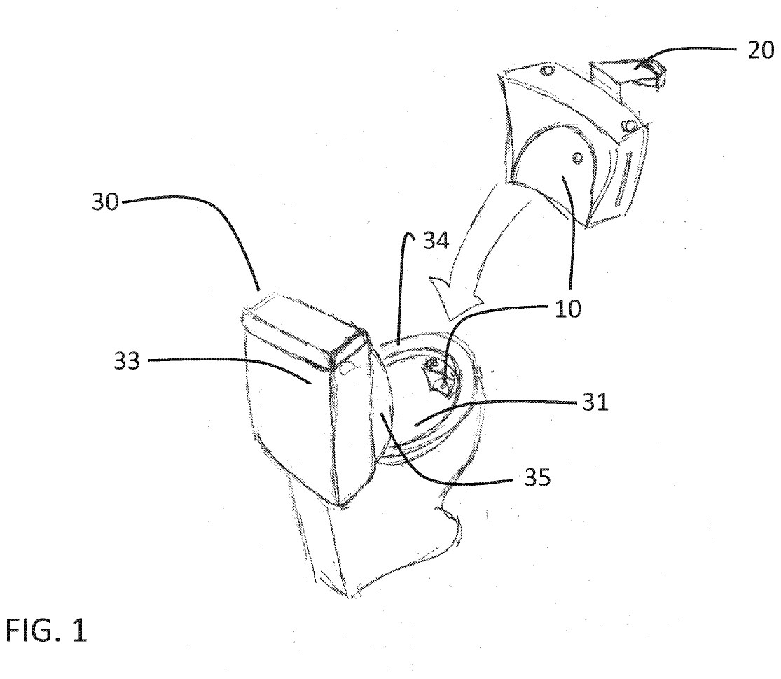

The toilet can be a toilet to which the detection unit can be attached. This includes, without limitation, Western toilets and squat toilets. In all cases the toilet will comprise a bowl for depositing excreta and a support to which the detection unit can be attached. In some embodiments the support can be the toilet bowl. Referring to FIG. 1, a modern Western flush toilet 30 typically comprises a vitreous, ceramic bowl 31 containing water and plumbing to fill the bowl with water and a drain remove material in the bowl. Water can be supplied to the bowl from a tank 33, typically positioned above the bowl. The bowl comprises a rim 34. The toilet typically has a seat 35 which can be positioned up and away from the seat or, by flipping the seat, positioned to be supported by the rim.

The tank 33 may be formed integral with a part of the toilet bowl 30. Alternatively, the tank 33 may be separately installed. The tank 33 is adapted to hold water and to discharge water into the toilet bowl 31 to clean the toilet bowl 31 with a flush of water. In typical flush toilets water is delivered to the tank causing a float to rise to the rising water. The float, in turn, controls a device to stop water from entering the tank once it reaches a certain level. Flushing the toilet typically involves turning a lever which opens a hole at the bottom of the tank allowing the water to flow from the tank to the bowl. The drain is connected to a trap positioned to maintain a predetermined amount of water in the bowl. Flushing the toilet increases the level of water in the ball above the level of the trap causing water to flow through the trap in into a sewer.

In a squat toilet, the bowl (also referred to as a pan) is typically positioned in the floor, and the user squats over the bowl to do their business.

B. Detection Unit

The detection unit comprises one or more detectors and a wireless transmitter to transmit signals detected by the detector. The detection unit typically also comprises a computer processor and computer memory typically comprising instructions executed by the processor for, e.g., having the transmitter wirelessly transmit information detected by the detector to a receiver. The detection unit also typically comprises a power source, such as a battery. The detection unit optionally can include a motion sensor 47 to activate the detector.

Placed within the bowl of any ordinary toilet, the remote, in toilet, the detection unit will record the flow of excreta into the toilet bowl. FIG. 1 shows an exemplary detection unit 10 comprising support 20 that functions to attach the detection unit to a toilet 30. The support 20 can be a clamp, a hanger, a hook, a suction cup, a magnet, a bolt, a screw, a bracket or other suitable devices for attaching the unit to the toilet. As depicted in FIG. 1, exemplary support 20 comprises a hook or bracket that hangs over a rim of the toilet bowl, thereby attaching the detection unit to the toilet.

Once the detection unit has been mounted to the toilet bowl 31, a toilet seat 35 is hinged to the toilet bowl 31 and is rested on the rim of the toilet bowl 31, and a lid cover (not shown) is hinged to the rear side of the toilet seat.

In one embodiment, an excreta flow detection unit includes one or more detectors (such as a laser triangulation sensing device, infrared sensing device and/or Doppler velocimeter), a data processor, a data storage device, a wireless transmitter to transmit data from the infrared sensing device to a receiver that is connected to a communications network, a power source, and a support for mounting the unit in a toilet bowl.

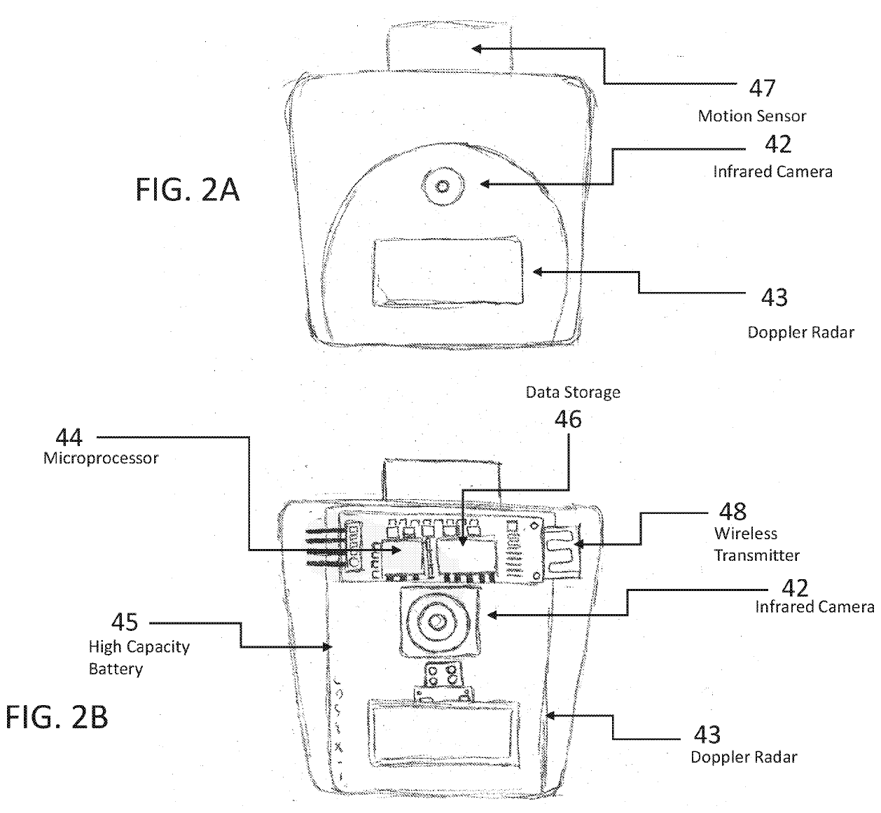

Viewing FIG. 2A-2B together, components of an exemplary system include detection unit 10 that can be mounted in the toilet bowl 31. Inside the detection unit 10 is provided detectors, in this embodiment, an infrared video camera 42 and Doppler radar 43, sending data to a processor 44, which in turn stores data in memory or alternatively a solid state data storage device 46. Upon query by a mobile device 60, the processor 44 sends data using a wireless transmitter 48. The sensor and processor system is powered by a battery 45. Detection unit 10 optionally includes motion sensor 47. The processing computer will be hosted remotely and contain proprietary processing software.

The detector or detectors can use any of a number of detection methods. These include, for example, still or video photography, infrared photography, interferometry, Doppler velocitometry, sound recording, 2D or 3D laser triangulation and the like. The detector can comprise, for example, a laser triangulation sensor, an infrared video camera and/or a laser Doppler velocimeter. Infrared video cameras useful in the systems disclosed herein can be commercially obtained from, for example, FLIR Systems (Wilsonville, Oreg.), InfraTec (Dresden, Germany), Allied Scientific Pro (Taipei City, Taiwan). Laser Doppler velocimeters are available from, for example, OptoMET (Darmstadt, Germany). Laser triangulation sensors are available from, for example, Wenglor Sensoric GmbH (Tettnang, Germany), Acuity Schmitt Industries (Portland, Oreg.), Keyence Corporation (Itaska, Ill.), Micro-Epsilon (Ortenburg, Germany) (e.g., optoNCDT).

In one embodiment, the detector can detect infrared radiation. All objects above the absolute zero temperature (0° K) emit infrared radiation. One way to measure thermal variations is to use an infrared vision device, usually a focal plane array (FPA) infrared camera capable of detecting radiation in the mid (3 to 5 μm) and long (7 to 14 μm) wave infrared bands, denoted as MWIR and LWIR, corresponding to two of the high transmittance infrared windows.

In passive thermography, the features of interest are naturally at a higher or lower temperature than the background. Passive thermography has many applications such as surveillance of people on a scene and medical diagnosis (specifically, thermology).

In active thermography, an energy source is required to produce a thermal contrast between the feature of interest and the background. The active approach is necessary in many cases given that the inspected parts are usually in equilibrium with the surroundings.

In an exemplary embodiment, detector 42 preferably uses passive thermography. With passive thermography, the features of interest are naturally at a higher or lower temperature than the background. Urine and feces exit the body at temperatures between 90° and 100° F. Ambient room temperature is 68° to 77° Fahrenheit. This temperature differential allows for thermal measurement of toileting in real-time, to produce flow rate and volume. It will also allow for graphic representation of flow patterns. In addition to improvement and replacement of existing clinical technology, this approach enables physicians and caregivers to know if patients are chronically constipated, afflicted with diarrhea, or are seeing reduced urine flow. With this data, interventions can be undertaken to prevent severe dehydration and potential hospitalization.

In one embodiment, the system includes a remote infrared technology that accurately captures thermal flow in real time. The processors within the device must record and utilize local storage to hold and time stamp data for upload. The processor 44 and software within the device can utilize wireless technology to upload data to a central processing server. A cloud based server and software can interpret and report encounter with high accuracy. The reporting may include imaging of specific patterns and playback for clinical review.

In another embodiment, the detector can comprise a laser Doppler velocimeter (“LDV”). Laser Doppler velocimeters detect displacement and velocity using lasers to detect Doppler shifts of moving objects. A split laser beam is directed to a moving flow stream. Reflected light forms an interference or fringe pattern. The interference pattern indicates Doppler effect which, in turn, indicates velocity and displacement.

In another embodiment, the detection unit employs projected or structured light 3D scanners. Historically known as “white light” 3D scanners, most structured light 3D scanners today use a blue or white LED projected light. These 3D scanners project a light pattern consisting of bars, blocks or other shapes onto an object. The 3D scanner has one or more sensors that look at the edge of those patterns or structure shapes to determine the objects 3D shape. Using the same trigonometric triangulation method as laser scanners the distance from the sensors to the light source is known.

In another embodiment, the detection unit employs laser triangulation detection. Laser triangulation can use 2D or 3D detection. In laser triangulation, structured light, such as laser light is shined on an object. Laser triangulation scanners generally use semiconductor lasers due to their low cost and their small size. Reflected light is captured by a camera, such as a CCD camera. Distance from the laser to the object is determined by principles of trigonometric triangulation using the distance between the camera and the laser and the angle at which the light reflected from the object strikes the camera. The two-dimensional shape of an object can be determined by projecting a line of laser light onto the object. On contact with the object the line deforms and is reflected back to the camera. The three-dimensional shape of an object can be determined either by moving the object across a line of laser light, referred to as 2D detection, or by projecting a plane of light on the object. The camera captures millions of points which can be reconstructed into a 3D shape. 2-D detection is enhanced by placing a plurality of detectors around a three-dimensional object and moving the three-dimensional object across the lines projected by the scanners.

Accordingly, the volume of moving excreta deposited into a toilet can be determined by 2D laser triangulation using a plurality of laser translation detectors. For example, three laser triangulation sensors can be positioned at about 120° angles in a circle. For laser triangulation sensors can be positioned at about 90° angles in a circle. As the excreta moves past the lines of light projected by the detectors a three-dimensional image can be reconstructed and total volume extrapolated from this data. One such commercially available system to perform this is the Wenglor system. The system uses three or four sensors to examine an object from four angles. The measurements received are integrated to produce a three-dimensional creation of the object being measured. In the present methods, the volume of excreta is determined from this three-dimensional recreation.

Alternatively, one or more 3D laser triangulation scanners can be used to produce the same result.

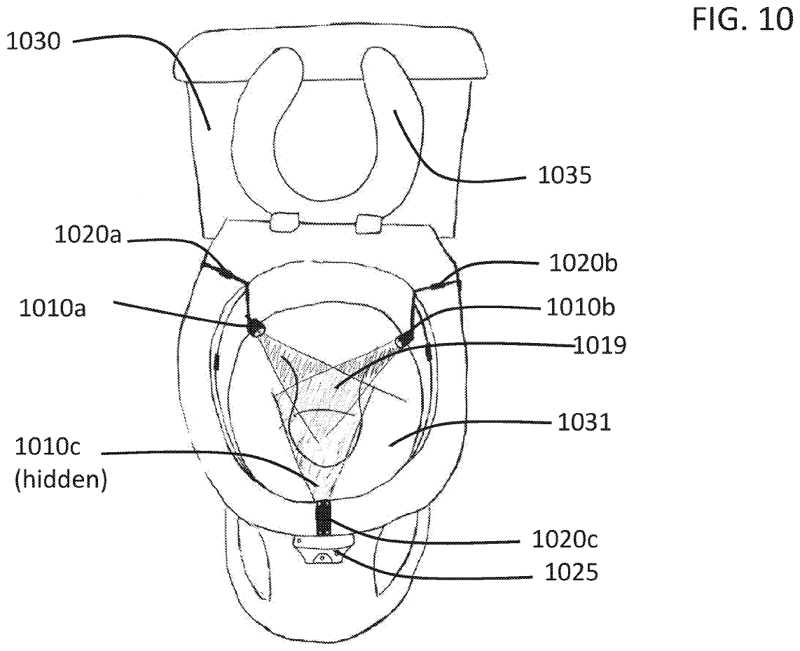

Referring to FIG. 10, toilet 1030 comprises a seat 1035 and a bowl 1031. The detection unit comprises three lasers 1010 a, b, and c (hidden) that project, in cross section, lines of laser light, e.g., 1019. The lasers are held in place by brackets 1020 a, b and c that function to attach the detection unit to a toilet 1030. Transmitter 1025 transmits information collected by the laser sensors.

In one embodiment, the detection unit comprises a combination of two or more of laser triangulation sensing, Doppler radar and/or an infrared detector. Doppler radar can be used to detect displacement and velocity of excreta deposited into the toilet bowl. Laser triangulation can be used to detect shape of the excreta. The infrared detector can detect heat information from the excreta. Together, these detectors can determine the kind of excreta and its amount over time for calculation of flow rate and amount.

The detectors are positioned to detect excreta deposited into the toilet bowl 31 and passing through a data capture field 71.

The detection unit can comprise a motion sensor to sense commencement of toileting activity. In certain embodiments, the sensor can be configured to sense motion near the toilet, for example, a person standing in front of or sitting down on the toilet. In other embodiments the motion sensor can be configured to detect motion of material being deposited into the toilet bowl.

Data collected by the detector is wirelessly transmitted by a transmitter in the detection unit to a remote receiver. Optionally, the data may be preprocessed before transmission. Preprocessing can include, for example, data compression.

Receiver/Communications Network

Measurement data from the detector will be sent via existing wireless technology through a communications network to a central processing server for interpretation and reporting. This approach allows for lowest possible cost of the remote solution with maximum computing power for accuracy.

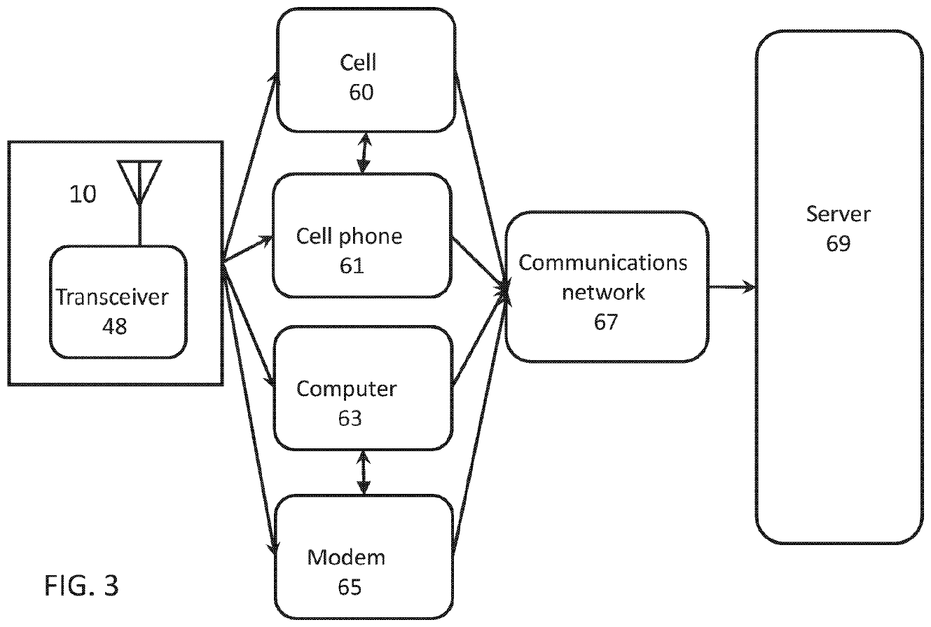

Referring to FIG. 3, the communications network can be any available network that connects to the Internet. The communication network can utilize, for example, a high-speed transmission network including, without limitation, Digital Subscriber Line (DSL), Cable Modem, Fiber, Wireless, Satellite and, Broadband over Powerlines (BPL). Accordingly, transceiver 48 can be configured for short link communication, e.g. Bluetooth, and can connect to a receiver such as a cellular telephone 61 which connects through a cell 60 to a cellular telephone communications network 67 or a computer 63 which connects to a communications network by Wi-Fi or by a direct wire connection. Alternatively, transceiver 48 can connect by Wi-Fi to a local area network 65 which is connected to the communications network 67. In another embodiment, transceiver 46 connects directly to a cellular telephone network through a cell connection. Communications network 67 transmits received signal to a remote server 69.

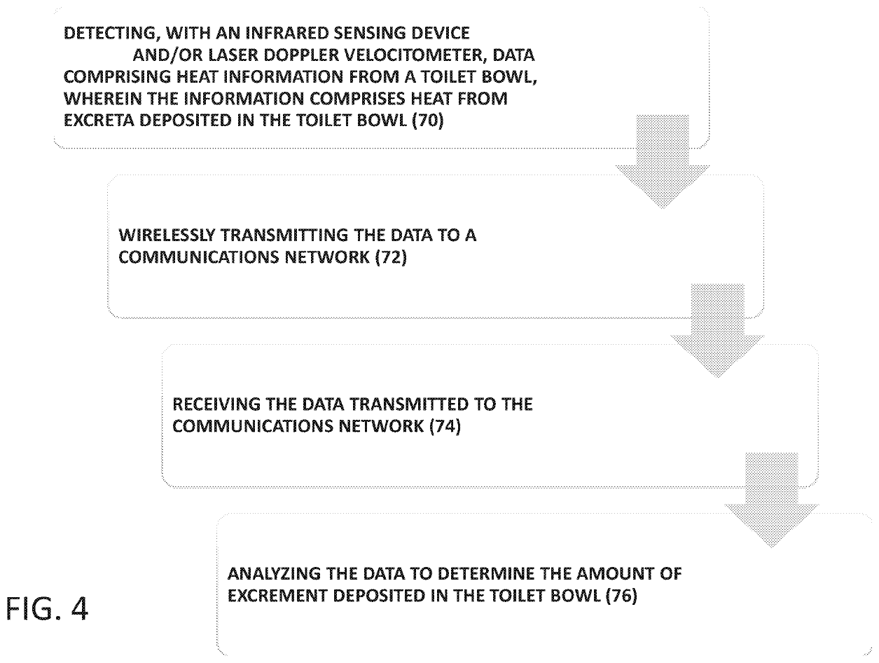

FIG. 4 shows an exemplary process executed by the processor 44. The process can be performed by executing computer code for detecting, with an infrared sensing device, data comprising heat information and/or Doppler radar from a toilet bowl, wherein the information comprises heat from excreta deposited in the toilet bowl (70). The code can also include wirelessly transmitting the data to a communications network upon demand (72). The network can include a mobile device such as a smart phone to display results, or can include a server with code for receiving the data transmitted to the communications network (74) and analyzing the data to determine the amount of excreta deposited in the toilet bowl (76).

Data relating to measurement of flow rate, volume, and structure of void can be transmitted via Bluetooth, WiFi or cellular network to central server for processing. Clean, accurate, cost effective measurement and monitoring of toileting and uroflowmetry both in clinical and home settings can be done, which is not a possibility with currently available technologies.

The excreta analyzed can be urine and/or feces. The detection unit 10 has a support 20 (FIG. 1) which can be mounted to the bowl 31 by means of a clamp, a hanger, a hook, a suction cup, a magnet, a bolt, a screw, a bracket or suitable other devices for attaching the unit to the toilet. The infrared sensing device can be an infrared camera. The data storage device can be instructions for converting a signal from the infrared sensing device for transmission by the wireless transmitter. The data processor executes instructions from the data storage device for converting a signal from the infrared sensing device for transmission by the wireless transmitter. The wireless transmitter is configured for short link radio communication (e.g., Bluetooth); communication over a wireless local area network (e.g., Wi-Fi) or communication over a cellular cell phone). The power source can be a battery. The amount of urine and/or feces is determined as a function of heat measurements included in the data. The computer can run code analyzing the data analyzes a video image in which urine is detected at a first temperature in feces is determined at the second temperature. The computer code can include analyzing the data detection urine and/or feces as a key difference above ambient temperature. The computer code can include analyzing the data analyzes data collected over a predetermined time, e.g., about one hour to about one day, about one day to two weeks, about one week to about one month, about one month to about one year.

### III. Computer System

Data is transmitted over the communications network to a server where it is available for access and analysis by a computer comprising analytic software.

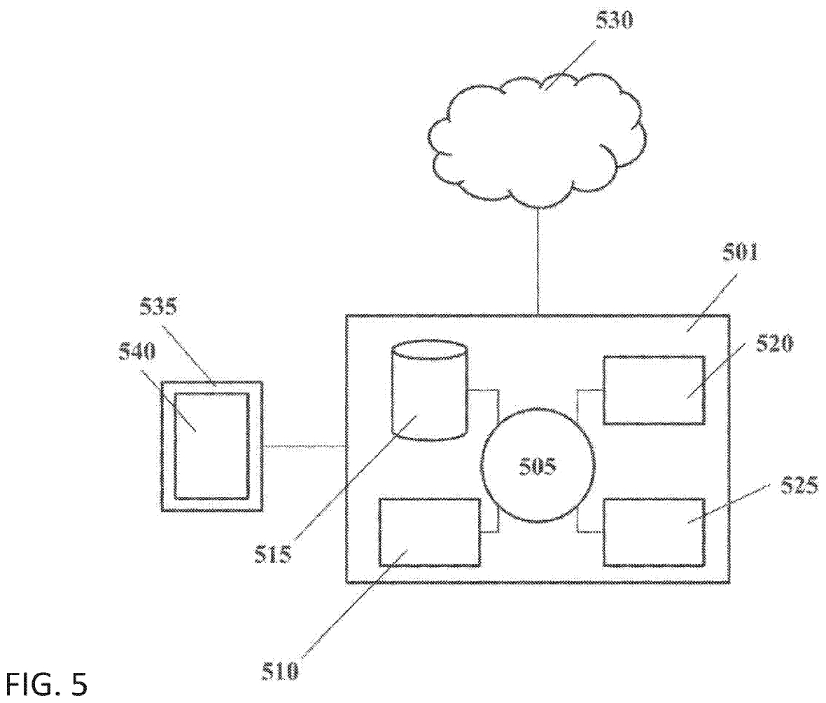

FIG. 5 shows an exemplary processing system. The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure.

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks.

The computer system 501 can include or be in communication with an electronic display 535 that comprises a user interface (UI) 540 for providing, for example, input parameters for methods described herein. Examples of UIs include, without limitation, a graphical user interface (GUI) and web-based user interface.

### IV. Methods of Evaluating Voiding Function

The use of infrared or thermal technology and/or Doppler velocimetry in the diagnostic assessment of voiding dysfunctions allows the system to replace urodynamic testing solutions and enable new, innovative, less invasive diagnostic measurement. It can replace current uroflowmetry technology and paper based bladder and bowel diaries. These diaries manually record toileting activity.

During the test, the subject can urinate or defecate into the toilet diagnostic system of FIG. 1 for the test. The patient can stand or sit on the seat and thus can urinate as he or she normally would, without attempting to manipulate the speed or flow in any way. The sensors generate data for the process of FIG. 4 to detect the speed and quantity of urination and defecation. In one embodiment, the using information transmitted by the detector of FIG. 1, an algorithm executed by a computer or person calculates the amount of urine passed by the patient, the flow rate in seconds, and the length of time it takes to empty a bladder completely. It will record this information on a chart. During normal urination, the initial urine stream begins slowly, speeds up, and then finally slows down again.

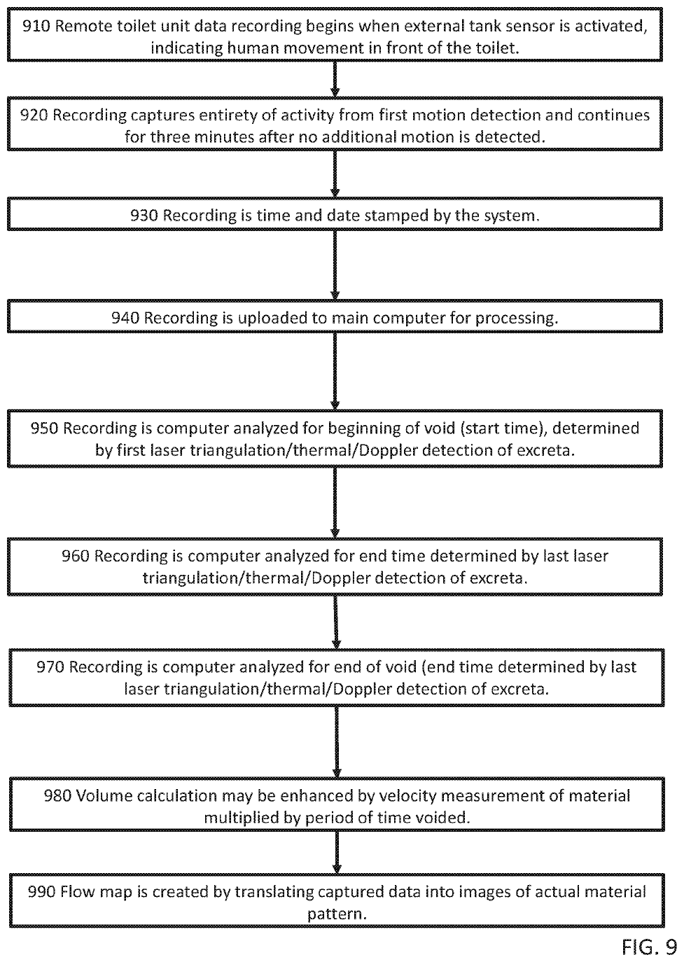

In an embodiment, depicted in FIG. 9, remote toilet unit data recording begins when external tank sensor is activated, indicating human movement in front of the toilet 910. Recording captures entirety of activity from first motion detection and continues for three minutes after no additional motion is detected 920. Recording is time and date stamped by the system 930. Recording is uploaded to main computer for processing 940. Recording is computer analyzed for beginning of void (start time), determined by first laser triangulation/thermal/Doppler detection of excreta 950. Recording is computer analyzed for end of void (end time) determined by last thermal/Doppler detection of excreta 960. Recording is analyzed for volume of void by measurement of diameter of voided excreta material in predetermined data capture field and multiplying volume by period of time voided 970. Volume calculation may be enhanced by velocity measurement of material multiplied by period of time voided 980. Flow map is created by translating captured data into images of actual material pattern 990.

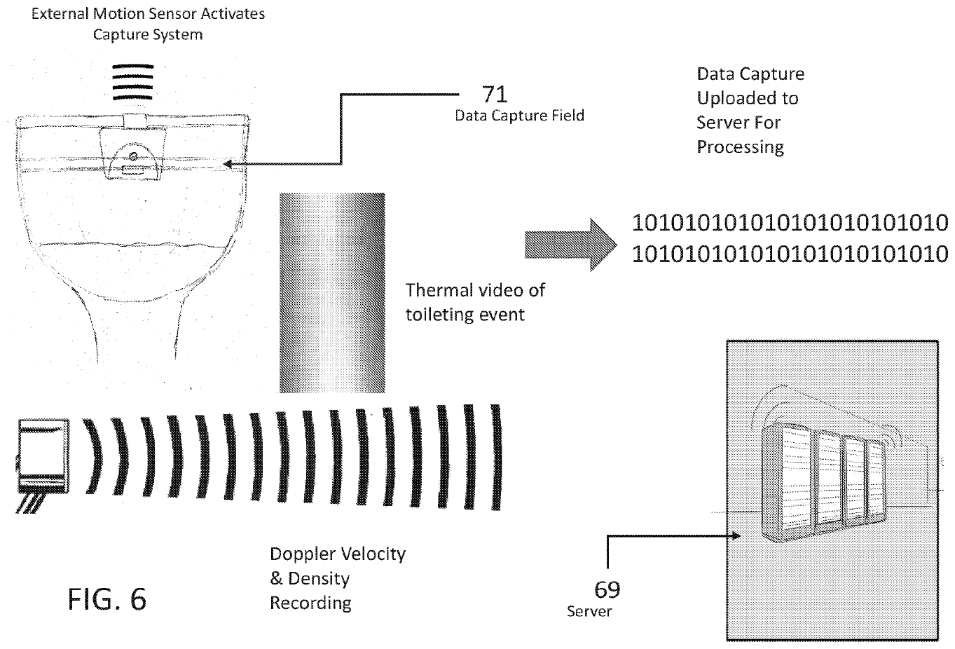

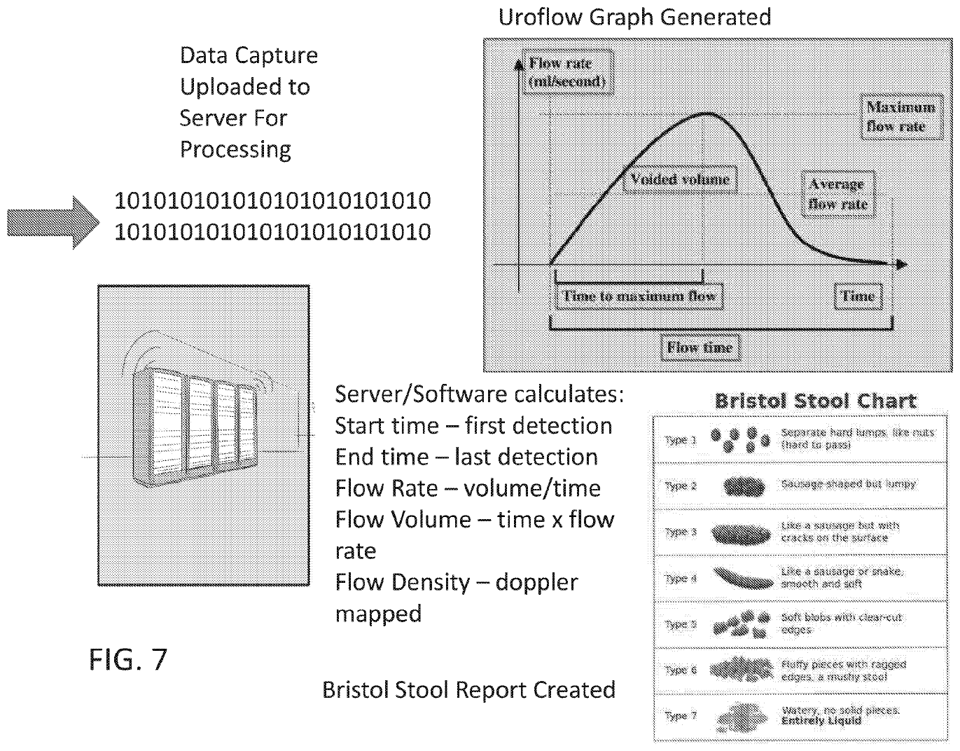

One embodiment of the process is depicted in FIGS. 6 and 7. The motion detector detects commencement of voiding activity, activating the detection system. The detectors, in this embodiment in infrared video camera and a laser Doppler velocimeter, measure singles from a data capture field 71 and transmit the data through the communications network to server 69. Using an algorithm, as described herein, a computer or a human operator analyzes the data. Results can be depicted, for example, as a uroflow graph and/or a Bristol stool report.

Typically, measurements are made over a predetermined time period such as at least 12 hours, at least one day, at least 3 days, at least one month or a plurality of months. The detector can record any differences from the norm to help a health care provider make a diagnosis. Results from the test can help a doctor determine how well the bladder and sphincter are functioning. It can also be used to test for obstructions in the normal flow of urine. By measuring the average and maximum rates of your urine flow, the test can estimate the severity of any blockage or obstruction. It can also help identify other urinary problems, such as a weak bladder or an enlarged prostate. All of this can be done in a convenient and clean method for patients and providers.

Based on the results of the analysis, a health care provider can provide a therapeutic regimen to the subject. A therapeutic regimen can involve drinking a specified daily intake of water. It may involve administering to the subject a pharmaceutical to treat a pathologic state identified by the test results. This may include, for example, providing an anti-diarrhea medication, or administration of antibiotics for treatment of urinary tract infection or prescribing bladder training to increase capacity and decrease frequency of micturition.

FIG. 6 depicts an exemplary process involving making measurements on excreta by both infrared video and LDV, and transmission of the data generated to a server while FIG. 7 depicts an exemplary process of analyzing data provided to a server and generating reports including, e.g., a uroflow graph and a Bristol stool report. Turning now to FIG. 6, an external motion sensor activates a capture system with a data capture field 71 such as the system of FIG. 1. In this embodiment, a thermal video of the voiding function is captured, and the data is uploaded to a server 69 for processor. In addition, a Doppler velocity and density recording is captured and sent to the server 69. FIG. 7 shows an exemplary processing at the server 60. The Server/Software calculates:
Start time—first detection
End time—last detection
Flow Rate—volume/time
Flow Volume—time x flow rate
Flow Density—Doppler mapped

A Bristol Stool Report can be generated by the server 69.

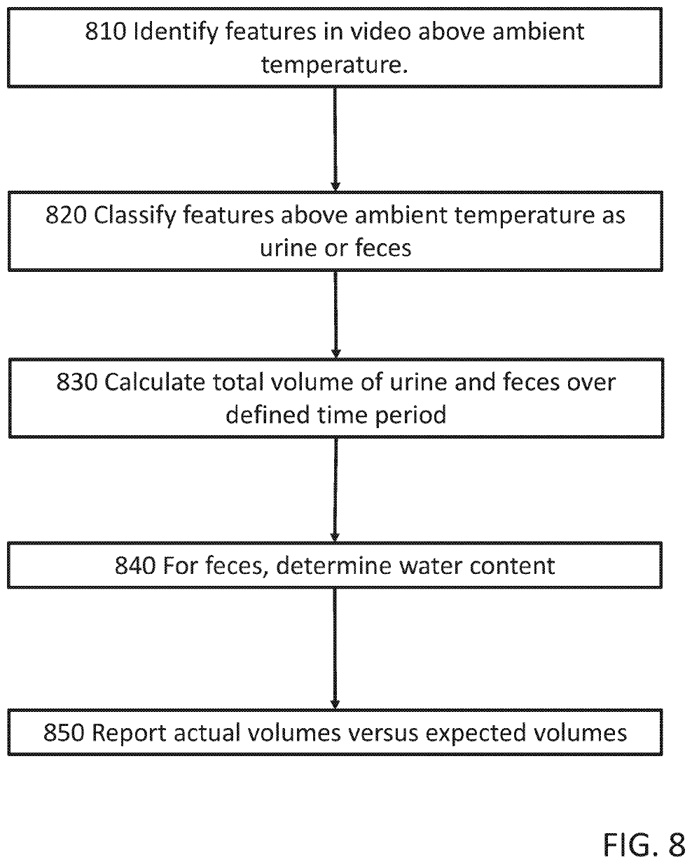

FIG. 8 provides a flow chart of an exemplary algorithm executed by a computer to analyze voiding data transmitted to the server. In step 810 the algorithm identifies features in an infrared video having a temperature above ambient. This is used to distinguish human excreta from other material. Human excreta will have a temperature of between about 90° F. and 100° F. In this step 820 features above ambient temperature are further classified as urine and or feces. This can be done based on shape and/or density. Generally, a stream of urine has a more narrow and regular shape than feces. In step 830, total volume or amount of feces and urine is determined. This volume or amount can be determined for each voiding event. The algorithm determines times of detection of beginning and end of a voiding event to calculate total time of the voiding event. Using information regarding velocity and displacement of excreta produced, e.g., by a laser Doppler velocimeter, flow rate is determined. Flow volume can be determined as a function of time and flow rate. Flow density also can be determined from the LDV data. In step 840 water content of feces is determined, for example, as a function of density. Classification of feces can be determined as a function of shape and density. Feces can be classified using the Bristol stool chart. The Bristol stool chart classify school into seven types. These types are further described in FIG. 7. Feces can be distinguished from urine because feces is denser than urine. In step 850 he reported generated detailing amount and kinds of excreta over time. In certain embodiments, the report can further comprise information regarding expected amounts and deviation in the individual from the expected amounts.

The analytic software comprises code that when executed differentiates liquid from solid waste in the transmitted data and quantifies the amount of each. Quantifying can include determinations of volume or mass, over a measuring period. Information can be presented as quantity as a function of time.

The analytic process needs to differentiate between solid and liquid wastes. An automatic detection algorithm, e.g., for infrared uroflowmetry can be used. After analyzing the characteristics of infrared images of bodily excretions, the method firstly designs Difference of Gaussians (DoG) filters to compute a saliency map. Then the salient regions where the potential targets exist in are extracted by searching through the saliency map with a control mechanism of winner-take-all (WTA) competition and inhibition-of-return (IOR). At last, these regions are identified by the characteristics of the excretion IR targets, so the true targets are detected, and the spurious objects are rejected.

In another embodiment laser triangulation methods produce a three-dimensional image of excreta. Based on the three-dimensional shape of excreta, software classifies the excreta as urine or feces. For example, a thin stream shape is classified as urine while a wider, possibly irregular shape is classified as feces. The software calculates volume based on the dimensions of the three-dimensional shape and the classification of the excreta. In another embodiment laser triangulation data is combined with Doppler velocity and density data. For example, data from Doppler measurements is used to determine whether excreta is urine or feces and the relative water content of each. Laser triangulation data is used to determine shape and volume of excreta the data is combined to determine volumes of urine and feces and to distinguish loose feces from dents feces based on density.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

What is claimed is:

1. A system comprising:
a) a toilet comprising a toilet bowl for receiving excreta;
b) a detection unit mounted to the toilet bowl and comprising: (i) one or more detectors selected from a group consisting of one or more laser triangulation sensors, an infrared sensing device and a Doppler velocimeter), (ii) a data processor, (iii) a data storage device, (iv) a wireless transmitter to transmit data from the detectors to a communications network and (v) a power source, wherein the one or more detectors are positioned measure characteristics of excreta deposited in the toilet bowl; and
c) a computer configured to receive the data over the communications network and comprising memory including computer code to analyze the data and determine an amount of urine and/or feces deposited into the toilet bowl, and a processor to execute the code.

2. The system of claim 1, wherein the toilet is a sitting toilet or a squat toilet.

3. The system of claim 1, wherein the excreta comprises urine and/or feces.

4. The system of claim 1, wherein the detection unit is mounted to the bowl by means of a hook, a clamp, a hanger, a suction cup, a magnet, a bolt, a screw or a bracket.

5. The system of claim 1, wherein the detector comprises one or more laser triangulation sensors.

6. The system of claim 5, wherein the one or more laser triangulation sensors comprise one or more 2D or 3D laser triangulation scanners.

7. The system of claim 1, wherein the detector comprises an infrared sensing device.

8. The system of claim 1, wherein the detector comprises a laser Doppler velocimeter.

9. The system of claim 1, wherein the detector comprises an infrared sensing device and a laser Doppler velocimeter.

10. The system of claim 1, wherein the detector comprises one or more laser triangulation sensors and a laser Doppler velocimeter.

11. The system of claim 1, wherein the data storage device comprises instructions for converting a signal from the infrared/laser sensing device for transmission by the wireless transmitter.

12. The system of claim 1, wherein the data processor executes instructions from the data storage device for converting a signal from the infrared/laser sensing device for transmission by the wireless transmitter.

13. The system of claim 1, wherein the wireless is configured for short link radio communication (e.g., Bluetooth); communication over a wireless local area network (e.g., Wi-Fi), communication over a cellular network or cell phone).

14. The system of claim 1, wherein the power source comprises a battery.

15. The system of claim 1, wherein the amount of urine and/or feces is determined as a function of heat measurements included in the data.

16. The system of claim 1, wherein the amount of urine and/or feces is determined as a function of 2D or 3D shape of the excreta.

17. The system of claim 1, wherein the computer code analyzing the data analyzes a video thermal image in which urine is detected at a first temperature in feces is determined at the second temperature.

18. The system of claim 1, wherein the computer code analyzing the data detects urine and/or feces as a function of temperature above ambient temperature.

19. The system of claim 1, wherein the computer code analyzing the data detects urine and/or feces as a function of velocity and/or displacement.

20. The system of claim 1, wherein the computer code analyzing the data detects urine and/or feces as a function of shape and/or density of excreta.

21. The system of claim 1, wherein the computer code analyzing the data analyzes data collected over a predetermined time, e.g., about one hour to about one day, about one day to two weeks, about one week to about one month, about one month to about one year.

22. A excreta flow detection and reporting unit comprising:
(i) one or more detectors selected from a group consisting of one or more laser triangulation sensors, an infrared sensing device and/or a laser Doppler velocimeter)
(ii) a data processor,
(iii) a data storage device,
(iv) a wireless transmitter to transmit data from the infrared sensing device to a receiver that is connected to a communications network,
(v) a power source, and, optionally,
(vi) a support for mounting the unit in a toilet bowl.

23. The unit of claim 22, wherein the support comprises a clamp, a hanger, a hook, a suction cup, a magnet, a bolt, a screw, or a bracket.

24. A method comprising:
a) detecting, with one or more detectors, excreta data from a toilet bowl by laser triangulation sensing, infrared sensing, Doppler velocity sensing, or a combination thereof;
b) wirelessly transmitting the data to a communications network;
c) receiving the data transmitted to the communications network into a server; and
d) analyzing the data to determine the amount of excreta deposited in the toilet bowl.

25. The method of claim 24, wherein the detecting further comprises activating the one or more detectors to collect data upon detection of motion with a motion sensor and deactivating the one or more detectors upon detection of cessation of motion with the motion sensor, wherein the motion is associated with excreta is being deposited in the toilet bowl.

26. The method of claim 24, wherein the detectors comprise an infrared video camera and a laser Doppler velocimeter, and analyzing the data comprises one or more of (i) duration of excretion (e.g., based at least in part on the first time in last time of detection of excreta through a data capture field), (ii) flow rate (e.g., based on volume per unit of time); (iii) flow volume (e.g., as a function of duration and flow rate); and (iv) class of excreta (e.g., urine or feces, e.g., based on density and/or shape of excreta).

27. The method of claim 24, wherein the detectors comprise one or more laser triangulation sensors, and a Doppler velocitometer, analyzing the data comprises:
determining three-dimensional shape and volume of excreta using data from the laser triangulation sensors;
determining density of the excreta using Doppler velocitometer data;
categorizing excreta as urine or feces based on density and/or shape data; and
determining volume and/or type of excreta based on shape and/or volume data and density data.

28. The method of claim 24, further comprising:
e) generating a report indicating an amount and pattern of urine and/or feces deposited into the toilet bowl over a specified period of time.

29. The method of claim 28, wherein the report comprises a uroflow graph and/or a Bristol stool report.

30. A system comprising:
a) a communication interface that receives, over a communications network, laser triangulation sensing data, infrared sensing data, Doppler velocity sensing data, or a combination thereof; and
b) a computer in communication with a communications interface, wherein the computer comprises one or more processors and a computer readable medium comprising machine executable code in tangible form that, upon execution by the one or more computer processors, implements a method comprising:
i) receiving the data, over the communications network;
ii) executing the machine executable code to determine volume and type of excreta, optionally including timing of excretion; and, optionally,
iii) transmitting the determinations over the communications network, e.g., to a remote computer.
